(19) United States
(12) Patent Application Publication
(10) Pub. No.: US 2024/0358438 A1
(43) Pub. Date: Oct. 31, 2024

(54) REMOTE PATIENT MONITORING AND CARE COORDINATION PLATFORM TO SUPPORT HYPERTENSION CARE OPTIMIZATION

(71) Applicant: Medtronic Ireland Manufacturing Unlimited Company, Dublin 2 (IE)

(72) Inventor: Kaitlin Chantal Templeton, San Francisco, CA (US)

(73) Assignee: Medtronic Ireland Manufacturing Unlimited Company, Dublin 2 (IE)

(21) Appl. No.: 18/649,578

(22) Filed: Apr. 29, 2024

Related U.S. Application Data
(60) Provisional application No. 63/462,997, filed on Apr. 29, 2023.

(51) Int. Cl. A61B 34/10; A61B 5/00; A61B 5/021; G16H 40/20; G16H 40/67

(52) U.S. Cl. CPC A61B 34/10; A61B 5/0004; A61B 5/021; G16H 40/20; G16H 40/67

(57) ABSTRACT

A system and method for remote monitoring of a hypertension patient including enrolling a hypertension patient in a remote patient monitoring application configured for a smartphone of the patient, receiving by the remote patient monitoring application patient blood pressure data from a blood pressure monitor, transmitting via the remote patient monitoring application the patient blood pressure data to a medical professional's application, receiving via the remote patient monitoring application one or more tasks from the medical professional's application, and transmitting results of the one or more tasks via the remote patient monitoring application to the medical professional's application.

### CROSS REFERENCE TO RELATED APPLICATION

This application claims priority to U.S. Provisional Patent Application No. 63/462,997 titled, A REMOTE PATIENT MONITORING AND CARE COORDINATION PLATFORM TO SUPPORT HYPERTENSION CARE OPTIMIZATION filed Apr. 29, 2023, the entire contents of which is incorporated herein by reference.

### TECHNICAL FIELD

This disclosure relates to systems and methods of remote monitoring of patients, identifying candidates for a medical procedure, and monitoring of the patient following the procedure.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, (i.e., renal dysfunction as a progressive complication of chronic heart failure). Pharmacologic strategies to thwart the consequences of renal sympathetic stimulation include centrally acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, have limitations and alternative therapies are desired.

### BRIEF DESCRIPTION OF THE DRAWINGS

FIG. 1A is a user interface of an application for remote monitoring of patients in accordance with aspects of the disclosure;

FIG. 1B is a user interface of an application for remote monitoring of patients in accordance with aspects of the disclosure;

FIG. 2A is a user interface for entry of patient data into an application for remote monitoring of patients in accordance with aspects of the disclosure;

FIG. 2B is a user interface for entry of patient data into an application for remote monitoring of patients in accordance with aspects of the disclosure;

FIG. 3A is a user interface of an application for remote monitoring of patients in accordance with aspects of the disclosure;

FIG. 3B is a user interface of an application for remote monitoring of patients in accordance with aspects of the disclosure;

FIG. 4 is a user interface for review of patient data in an application for remote monitoring of patients in accordance with aspects of the disclosure;

FIG. 5A is a user interface detailing a patient's profile in an application for remote monitoring of patients in accordance with aspects of the disclosure;

FIG. 5B is an alternative patient pathway indicator of FIG. 5A;

FIG. 5C is an alternative patient pathway indicator of FIG. 5A;

FIG. 6, is a user interface detailing patient data in an application for remote monitoring of patients in accordance with aspects of the disclosure;

FIG. 7 is a user interface detailing patient data in an application for remote monitoring of patients in accordance with aspects of the disclosure;

FIGS. 8A-8R depict a user interface of a smartphone application for remote collection of patient data in accordance with aspects of the disclosure;

FIG. 9 is an exemplary decision tree for assessing a patient's suitability for a denervation procedure in accordance with the disclosure; and

FIG. 10 is a schematic representation of a computing device configured to execute aspects of the disclosure.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a system for remote monitoring a patient, the system including a smartphone including a memory and a processor, the memory storing therein an application that when executed by the processor performs steps of: receiving patient blood pressure data from a blood pressure monitor; transmitting the patient blood pressure data to a medical professional's application; receiving one or more tasks from the medical professional's application; and transmitting results of the one or more tasks to the medical professional's application. Other embodiments of this aspect include corresponding computer systems, apparatus, and computer programs recorded on one or more computer storage devices, each configured to perform the actions of the methods.

Implementations may include one or more of the following features. The system where the application when executed by the processor receives vital sign data of the patient from a third-party application. The third-party application is a healthkit application configured to receive vital sign information from one or more connected devices. The application when executed by the processor receives and presents educational materials for the patient in a user interface on the smartphone. The application when executed by the processor transmits an indication of review of the educational materials to the medical professional's application. The application when executed by the processor presents the received blood pressure data in a user interface on the smartphone. The system may include a computing device including a memory and a processor, the memory storing therein the medical professional's application that when executed by the processor monitors the patient based on received results of the one or more tasks and received patient blood pressure data. The medical professional's application when executed by the processor determines at least in part by using or more of an algorithm, a neural network, or an artificial intelligence (AI), a patient's suitability for a denervation procedure. The medical professional's application when executed by the processor employs one or more of an algorithm, neural network, or ai analyze the received results of the one or more tasks, the received blood pressure data, and an electronic medical record of the patient to determine suitability for a denervation procedure. The medical professional's application when executed by the processor generates a referral of the patient to an interventional cardiologist (IC) for assessment of the patient for the denervation procedure. Implementations of the described techniques may include hardware, a method or process, or computer software on a computer-accessible medium.

A further general aspect of the disclosure includes a method for remote monitoring of a hypertension patient. The method includes enrolling a hypertension patient in a remote patient monitoring application configured for a smartphone of the patient; receiving by the remote patient monitoring application patient blood pressure data from a blood pressure monitor, transmitting via the remote patient monitoring application the patient blood pressure data to a medical professional's application, receiving via the remote patient monitoring application one or more tasks from the medical professional's application, and transmitting results of the one or more tasks via the remote patient monitoring application to the medical professional's application. Other embodiments of this aspect include corresponding computer systems, apparatus, and computer programs recorded on one or more computer storage devices, each configured to perform the actions of the methods.

Implementations may include one or more of the following features. The method may include receiving via the remote patient monitoring application educational materials; and transmitting to the medical professional's application and indicator that the educational materials have been reviewed. The method may include presenting a graph of the patient's blood pressure data on a screen in the remote patient monitoring application. The method may include monitoring at the medical professional's application the patient based on the received results of the one or more tasks and the received patient blood pressure data. The method may include determining via the medical professional's application suitability of the patient for a denervation procedure. The determining of suitability of the patient for a denervation procedure is made by hypertension clinic (HTN clinic) personnel. The method may include generating via the medical professional's application a referral of the patient to an interventional cardiologist (IC) for assessment of the patient for the denervation procedure. The determining of suitability of the patient is for the denervation procedure is performed at least in part by one or more of an algorithm, a neural network, or an artificial intelligence (AI). The one or more of an algorithm, neural network, ai analyze the received results of the one or more tasks, the received blood pressure data, and an electronic medical record of the patient. The method may include transmitting via the remote patient monitoring application blood pressure data to the medical professional's application after performance of a denervation procedure. Implementations of the described techniques may include hardware, a method or process, or computer software on a computer-accessible medium.

### DETAILED DESCRIPTION

This disclosure is directed to systems and methods of remote monitoring of patients of a HTN Clinic and identifying patients that are likely to benefit from specific care plans, which may include nerve denervation procedure in which the sympathetic nerves enervating one or more organs (e.g., kidneys, liver, etc.) to treat hypertension and other diseases caused by over-activation of the SNS, and/or systems and methods of monitoring patients. One such procedure is a renal denervation (RDN) procedure, for example utilizing the Simplicity Spyral IM System offered by Medtronic.

Currently, some patients who are diagnosed with hypertension have their condition monitored and treated through a specialized hypertension clinic (HTN Clinic). However, many hypertension patients are cared for via other practices including general cardiology practices, primary care physicians, internists, nephrology practices, and others. Though aspects of the disclosure are described in the context of an HTN Clinic, the disclosure is not so limited, and the methods and systems described herein may be implemented in most medical practices without departing from the scope of the disclosure. At the HTN Clinic, the patient must schedule an appointment periodically (e.g., weekly, bi-monthly, monthly, etc.). The frequency of the appointment may be based on the severity of the hypertension, the patient's comorbidities, and related care plans, a change in how the patient is experiencing symptoms and others. These appointments are somewhat time-consuming for the patient and take up resources of the HTN Clinic, often utilizing clinic personnel for tasks that are well below their licensing capabilities. Most of these appointments focus on obtaining patient vital signs as well as lifestyle and medication adherence coaching. Those vital signs include one or more of blood pressure, heart rate, weight, blood oxygen saturation (SaO₂), patient assessment of, shortness of breath, self-reporting of activities, medication adherence, and side effects or incidents (e.g., falls), and others. These recurring patient appointments for vital sign checks limit the number of patients that a HTN Clinic can serve as each appointment is conducted by medical professionals at the HTN Clinic.

Though described herein in the context of an HTN Clinic, the methods and systems described herein can also be used by other hypertension-focused practices including general cardiology, primary care, nephrology and others that though not formal HTN Clinics, can nonetheless benefit from the remote patient monitoring and self-reporting systems described herein.

As will be appreciated, the collection of the vital signs of the patient by medical staff, though important to the patient, is not directly improving the condition of the patient. Further, owing to the regularity of the appointments, the strain on the medical staff is not insignificant and the HTN Clinic's ability to care for additional patients or patients in need of acute care can be severely limited.

Many patients, however, are very functional and many are not particularly old or infirm. Further, the near complete integration of smartphones into the daily lives of patients means that most patients are sufficiently technologically savvy and have grown accustomed to utilizing a variety of applications on their smart phones to manage portions of their lives. These patients are good candidates for enrollment in a remote patient monitoring program in accordance with the disclosure. As described in greater detail below, with the receipt of patient vital sign data on a regular basis (often more frequent that traditional appointments) the HTN Clinic can conduct regular monitoring and triaging of patients. Thus, the remote monitoring reduces the frequency any single patient might need to visit the HTN Clinic and provides more accurate and granular patient data than could ever be achieved even by daily personal visits to an HTN Clinic.

In accordance with aspects of the disclosure the vital sign data described herein can be captured by a variety of tools that are already at the patient's disposal. For example, a mobile computing device, such as a smart watch or fitness watch (e.g., an APPLE® watch), a smartphone, or other device can capture a variety of vital signs including heart rate and blood oxygen saturation as part of their current tool set. Further, there are a variety of other devices capable of capturing vital sign data and communicating this information to a health application on the patient's mobile computing device, such as a smart phone (e.g., APPLE HealthKit, Google Fit, or others). In many ways, the vital sign data that is captured by these devices and tools mirrors that captured by doctors and nurses at the HTN Clinic. In accordance with aspects of the disclosure the vital sign data captured by the devices and tools can be analyzed by the patient via an application on their smartphone and can also be shared with other applications, such as those employed at the HTN Clinic.

Indeed, in many cases the data collected remotely may offer advantages over the data collected in the HTN Clinic. For example, cases of white coat hypertension or masked hypertension can only be detected with both in office and out of office measurements. Additionally, continuous data and/or more frequent measurements of blood pressure and other vital signs offer a greater depth of understanding of the burden of disease faced by a particular patient. Diurnal and seasonal variations in blood pressure are known to exist yet in-office measurements fail to capture the degree of variation in the case of a particular patient. Studies have shown a degree of variation of blood pressure within a single day, but this variation is not well captured by single point in time in office measurements. Thus, in accordance with the disclosure improvements in the standard of care for data acquisition are enabled by the collection of greater volumes of data for each patient. This data, when transmitted to applications at the HTN Clinic can be analyzed via algorithms (e.g., trained neural networks, artificial intelligence (AI) and others) to detect patterns in the data to alert the HTN Clinic medical professionals or the patient. Accordingly, aspects of the disclosure are directed to an application employed at the HTN Clinic while additional aspects of the disclosure are directed to a remote patient monitoring application. Further aspects of the disclosure are directed systems and methods of utilizing the data collected to identify patients that are likely to benefit from RDN procedures.

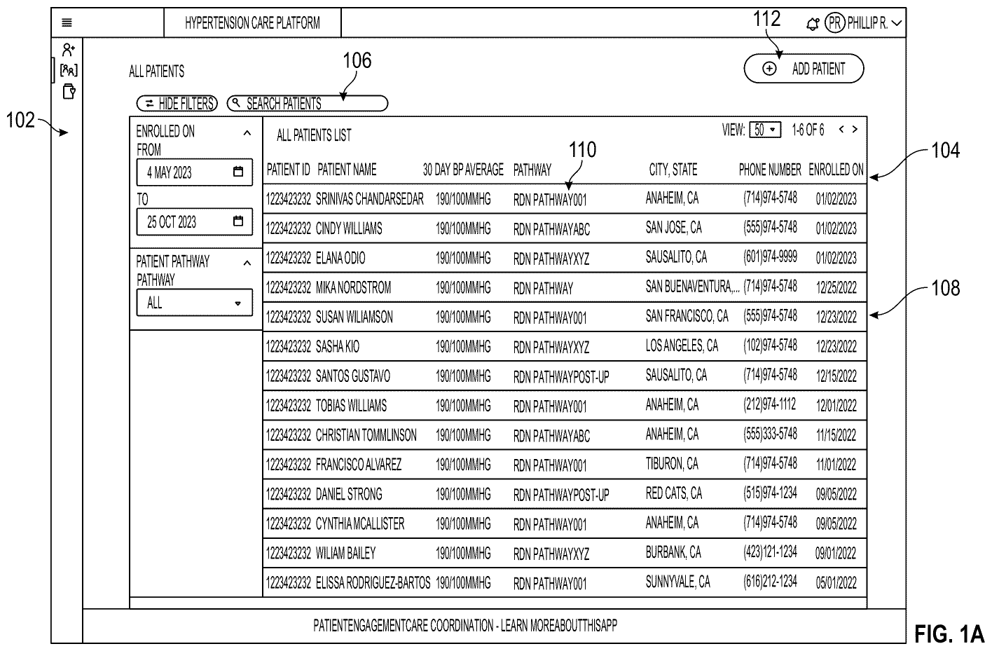

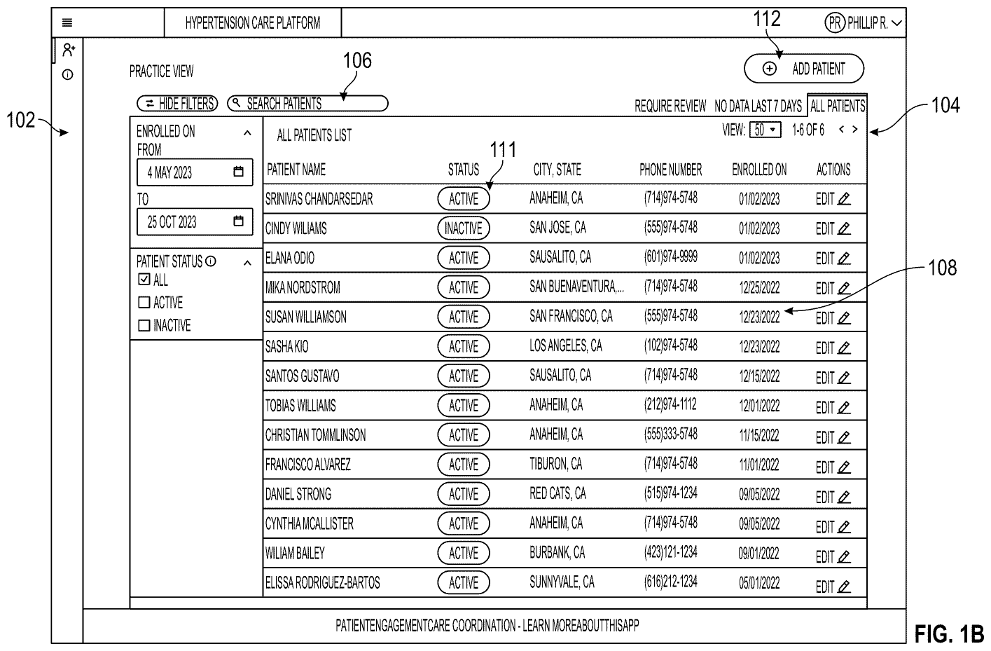

One aspect of the disclosure is directed to an application employed at an HTN Clinic to receive patient data from patients located remotely from the HTN Clinic, organize the patient data, and present the patient data in a useable and actionable form for ready analysis by healthcare professionals. FIG. 1A depicts a user interface 102 generated by an application either stored on and running on one or more computing devices, or accessible from a computing device, at the HTN Clinic. User interface 102 presents a table 104 of all patients enrolled in a remote monitoring program. The table 104 includes fields such as patient ID, patient name, 30-day blood pressure average, patient pathway, contact information, and enrollment date, as well as others. Further these fields may be selectively added or removed from table 104 to best accommodate the HTN Clinic practice. The user interface 102 includes a search filed 106 allowing a patient to be searched for by name. Enrollment date fields 108 indicates the date of enrollment of the patient and can be employed to reduce the number of patients whose data is presented in table 104. Similarly, a patient pathway field 110 can be used to segregate patients based on whether they are an RDN procedure candidate advancing a patient pathway (described in greater detail below). Each patient's entry on table 104 may include a hyperlink, either as part of the patient's name, or another filed for that patient in table 104.

FIG. 1B depicts an alternative user interface 102 where the patient pathway field 110 is replaced with a status indicator 111. The status indicator 111 depicts whether a patient is “active” or “inactive”.

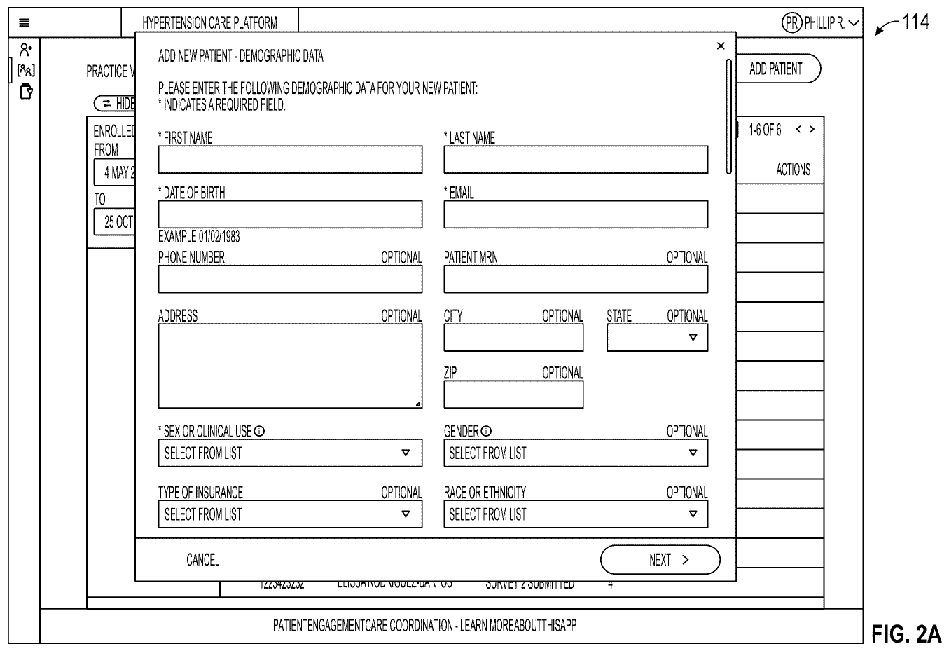

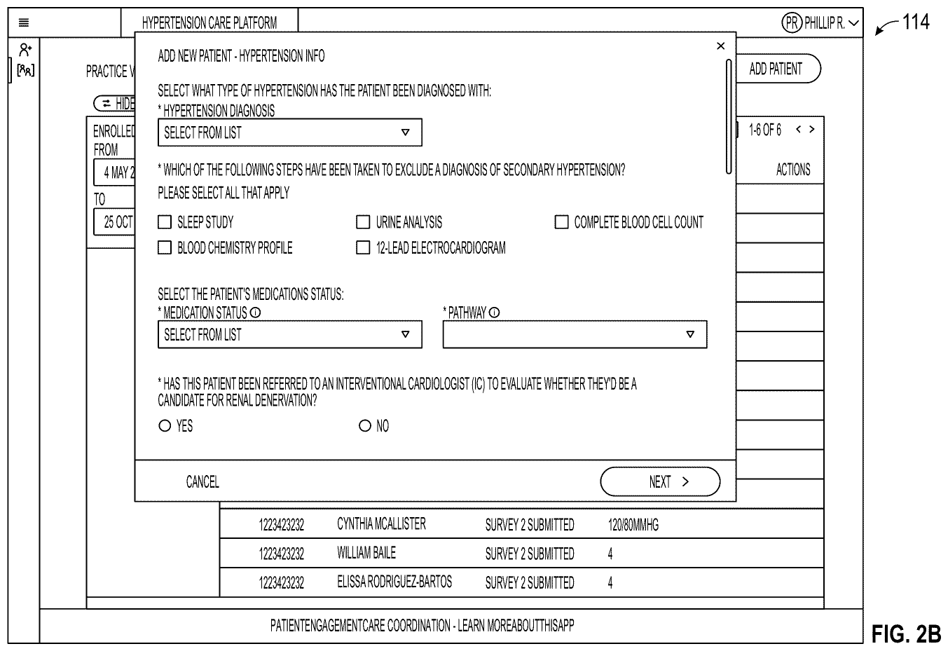

Selection of an “Add patient” tab 112 presents a fillable form 114, as shown in FIGS. 2A and 2B. By completing the fillable form and providing the relevant patient information an entry can be made for the patient in the application databases such that their data will appear in table 104 of FIG. 1. In FIG. 2A demographic information such as name, age, address, etc. is entered may be entered and captured by the application. FIG. 2B depicts a form 114 whereby additional hypertension data may be captured, such as the type of hypertension the patient has been diagnosed with whether the patient has undergone a sleep study, other diagnostic tests, medication status, whether the patient is enrolled on an RDN pathway, and whether the patient has been referred to an interventional cardiologist,

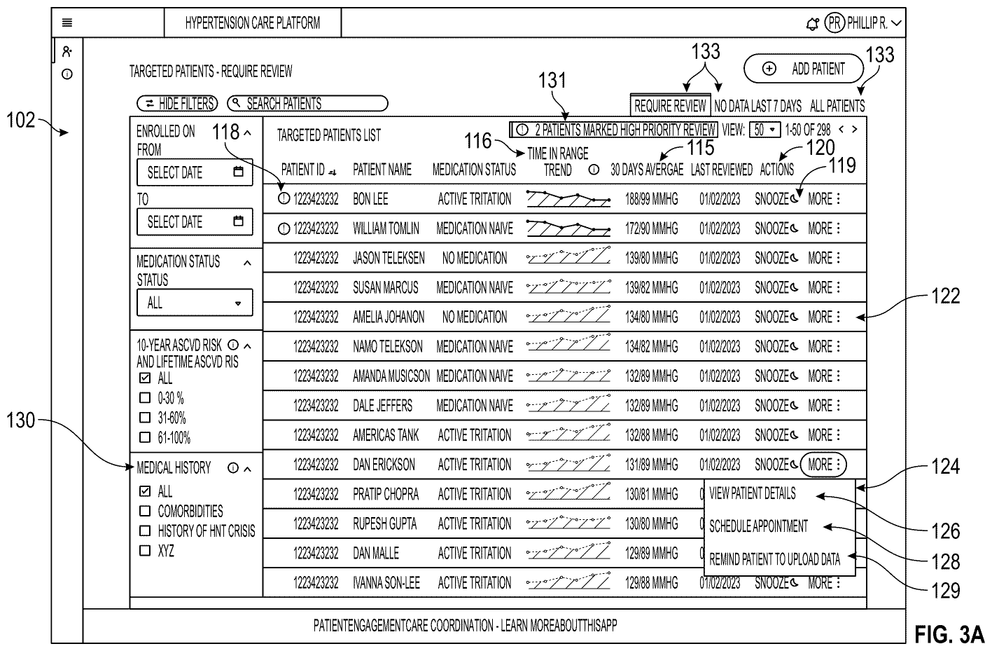

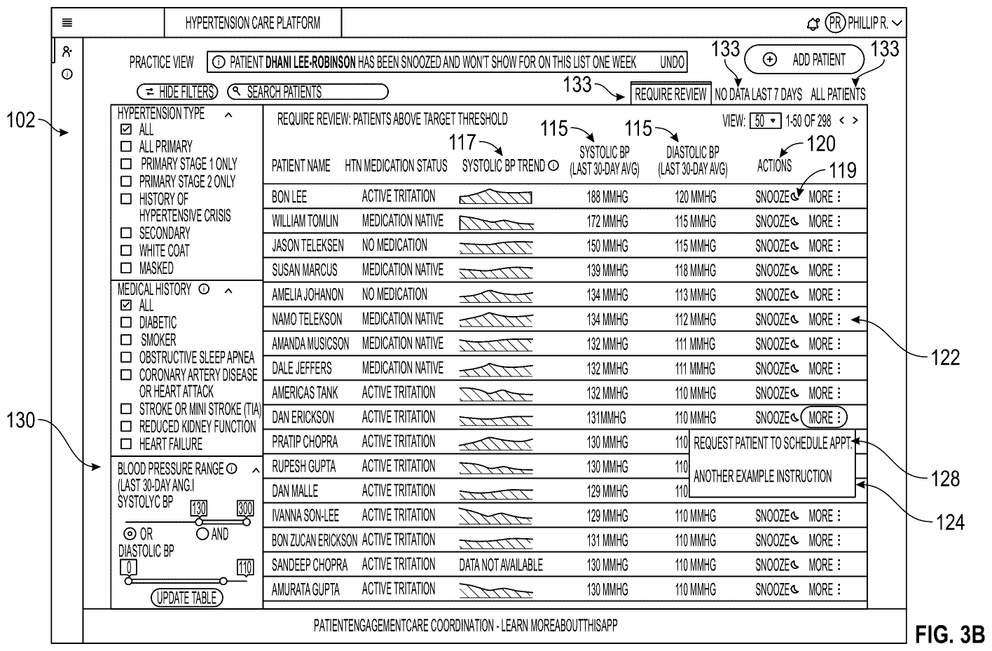

FIG. 3A presents the user interface (UI) 102 as it might appear to a clinician at the HTN Clinic when reviewing targeted patients. In FIG. 3A as a selectable alternative, in addition to the 30-day average blood pressure column of data 115, a “target-in-range” column 116 is presented. For each patient undergoing remote monitoring of their blood pressure, there may be a target range of blood pressure based on their individual disease state. The target range of blood pressure may be based on age, weight, comorbidities, medications, activity levels, and other factors. Once established, blood pressure data is remotely collected, and the data can be charted. A graph of the data may be presented in the target-in-range column 116 depicting the patient's data trend. Patients may be highlighted (e.g., where the patient's health is showing an indication of decline) by, for example, presenting the plot of the patient's blood pressure data in a different color, presenting the patient ID in a different color, presenting an indicator 118 in the table 104, or other means. Table 104 of FIG. 3A includes configurable columns data of the patient including medication status and a date of last receipt of patient health data, and others.

A variety of actions may be undertaken directly from user interface 102 by selecting additional links in the table. For example, for each patient listed in table 104 on FIG. 3, there is a “snooze” link 119 in the “actions” column 120. After reviewing a patient's data, a medical professional can assess whether the patient's data is acceptable, and selection of the “snooze” link 119 indicates that some review has been undertaken of the patient's data and no further action is required at this time. Using log-in credentials and the like the identity of the medical professional, as well as the time of the selection of the snooze link 119, and other actions related to the selection of the snooze link 119 can be captured and stored for later analysis if necessary.

Next to the snooze link 119 is a link 122 labeled “more.” Selection of the more link 122 produces a pop-up 124 where selection can be made from a variety of options including a “view patient details” link 126, a “schedule appointment” link 128 and a “remind” link 129 where an electronic reminder may be sent to the user (e.g., via text message, email, or via in-app communication, described below).

User interface 102 also provides other filters, which may be used by the medical professional to sort the data displayed. One such filter is the “medical history” filter 130. Another is a “Risk” value such as the Atherosclerotic Cardiovascular Disease (ASCVD) Risk value which provides 10-year risk of a patient developing ASCVD at an initial visit with a cardiologist. This provides a reference point for future visits and allows for forecasting of the effects of different interventions. This may be a stand-alone filter or incorporated with and a lifetime risk of ASCVD value. As will be appreciated, other filters may be employed without departing from the scope of the disclosure.

FIG. 3B provides an alternative user interface 102. Rather than depicting a “time-in-range” graph 116 a simple blood pressure trend graph 117 is depicted, this may be either the systolic blood pressure trend graph (as shown) or optionally a diastolic blood pressure trend graph. Similarly, the 30-day average blood pressure data 115 may show both the systolic and diastolic blood pressure averages. Additionally, the filters 130 include medial history, described above, hypertension type, and systolic and diastolic blood pressure ranges. Utilizing the filtering features, a patient list for review by the HTN clinic staff can be derived and presented on user interface 102.

As shown in FIGS. 3A and 3B, the patients are sorted based on a determined priority of review. Optionally as shown in FIG. 3A an indicator 131 of the number of patients for priority review may be displayed. This may be based on the last blood pressure data received, the trend of the patient's blood pressure data, rate of change of the trend, the blood pressure data in conjunction with other patient data (e.g., medications currently prescribed), and others. Regardless of the exact algorithm for sorting the patient hierarchy, those with the most pressing need for review may be shown at the top of table 104. This way a medical professional can make an immediate determination regarding the patient, based on the most recent data, that the patient may need acute medical intervention (e.g., ambulance services or taken to emergency room), less acute intervention (scheduling an appointment with the medical professional), or no action at this time. Thus, the use of remote monitoring of patient blood pressure relieves the HTN Clinic of the most labor intensive and least immediately informative portion of their practice.

Table 104 may include tabs 133 where patient data is sorted. A first tab 133 may include just those patients who based on the received vital sign and blood pressure data and for example a pre-set reporting frequency (e.g., bi-monthly) of that patient require review by the medical professional. Additionally or alternatively, the first tab 133 may depict just those patients expressing a blood pressure above a predetermined target blood pressure. The target blood pressure may be set by clinical guidelines or configured by the medical professionals for the specific patient taking into account the patient's specific factors (e.g., age, comorbidities, etc.) A second tab 133 may list just patients from whom no vital sign or blood pressure data has been received in a certain interval (e.g., the last 7-days). These patients may require the HTN Clinic to reach out to them to determine the reasons for the lack of vital sign data, and potentially scheduling an in-office visit. Yet another tab 133 may simply list all patients of the HTN Clinic.

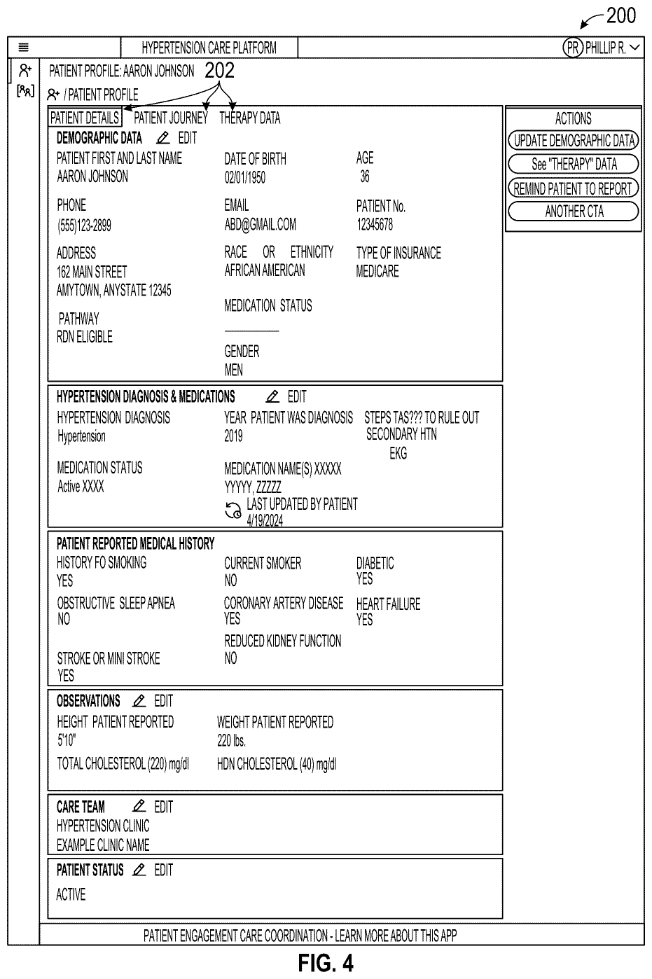

If the patient details link 126 is selected, or if the patient's name (with link) is selected the UI changes to UI 200, depicted in FIG. 4, where a patient profile is displayed. The Patient profile in UI 200 includes a number of tabs 202. A first tab 202 labeled “patient details” fields for the patient's name and contact information, and a care pathway. The care pathway may include, for example, whether they are on a path to or have already received renal denervation therapies and where they are on such pathway (as further described below).

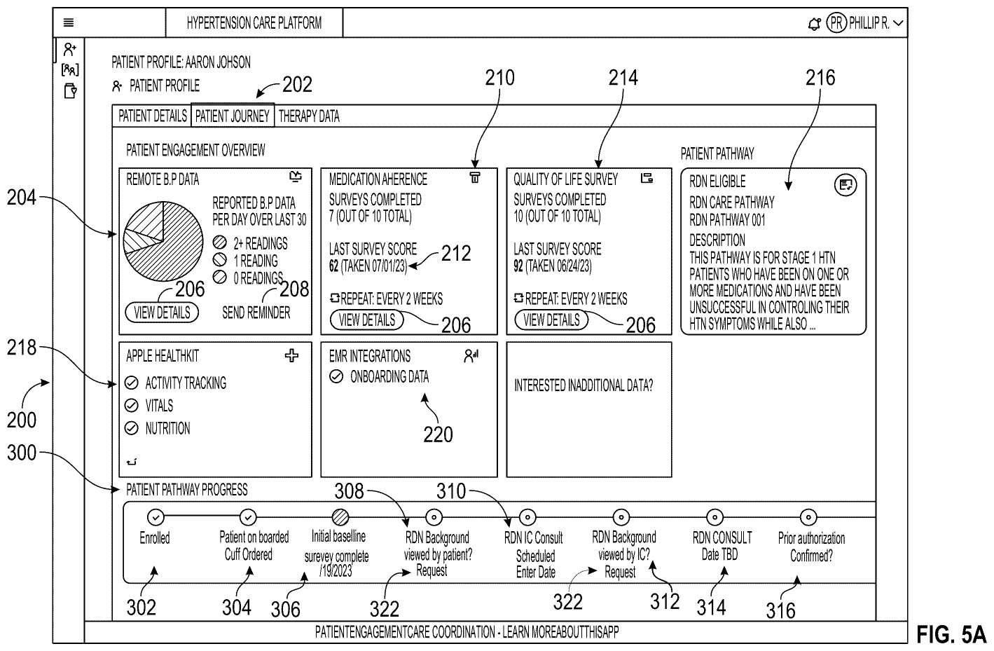

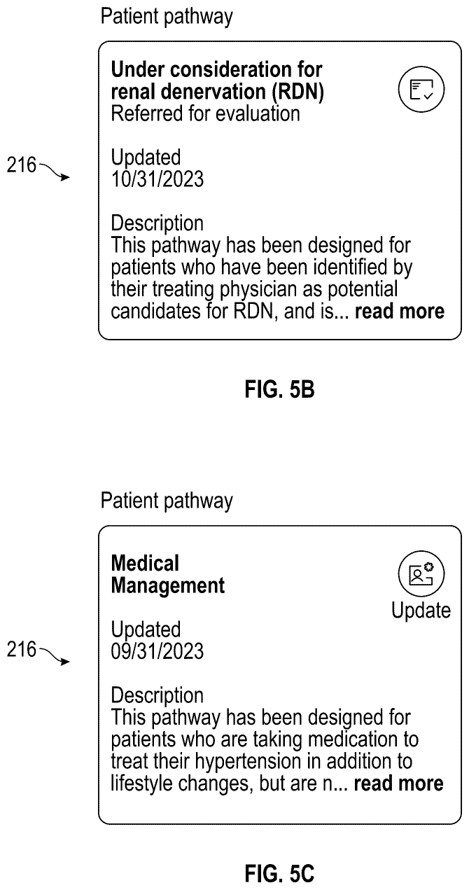

Selection of tab 202 labeled “pathway” changes UI 200 and presents the patient engagement overview as depicted in FIG. 5. The patient engagement overview can provide detailed data regarding the patient's compliance aspects of their monitoring and treatment plan. A remote blood pressure field 204 which provides an indication of the last 30 days of blood pressure data for the patient, as shown, the remote blood pressure data includes the number of remote blood pressure readings received each day of the preceding 30 days. More detailed information regarding the individual remote blood pressure by selecting a “view details” link 206, similarly, if desired a “send reminder” link 208 may be selected and a message (e.g., text, email, in-app notification, etc.) may be sent to the patient to remind the patient to record and transmit their blood pressure. A medication adherence field 210 provides a self-reported indication of medication adherence (e.g., how regularly the patient is taking their medications in accordance with their prescription). At the highest level, the medication adherence field 210 may merely provide an indication of how many surveys have been completed and returned to the application and a score 212 associated with the most recent survey. The details of the patient's response to the surveys may be reviewed by selecting the “view details” link 206 which can display the self-reported surveys of the patient. A quality-of-life survey field 214 displays information relating to the patient's responses to a self-reported survey of the patient's quality of life. Such surveys may include questions regarding mobility, breathing, energy levels, and the like. Again an indication of the number of surveys to which the patient has responded and a score 212 associated with the most recent survey may be presented in the quality-of-life survey field 214. Further details regarding individual surveys may be viewed by selecting the “view details” link 206.

A patient pathway field 216 provides a high-level indication of how the patient's hypertension is primarily being treated, which may include a combination of lifestyle modification, medication, a denervation procedure and/or other hypertension treatment options. A patient may be assigned to a pathway by their clinician, or a determination of eligibility may be made automatically by the application by accessing the patient data available to the application, including the patient's electronic medical record (EMR), as described in greater detail below. FIGS. 5B and 5C depict alternative patient pathway fields 216 in accordance with pathways a patient's hypertension may be managed. FIG. 5B indicates that the patient is under consideration for an RDN procedure, whereas FIG. 5C indicates that the patient's hypertension is being medically managed.

Additional details of the patient are provided in a Health Kit field 218 which details the patient's connectivity via one or more applications on their mobile phone and connected to various sources of data including blood pressure cuffs, activity tackers, vital sign data collectors (e.g., Apple® Watch). and nutrition trackers where patients can detail their caloric intake (e.g., food and drink consumption). An EMR integration field 220 identifies whether access has been established to the patient's EMR. Additional fields may be added as desired by the medical professional.

A patient pathway progress indicator 300 displays where the patient is on their pathway to receiving a renal denervation (RDN) therapy as a means of quickly bringing the clinician up to speed on the patient's care plan progress and displays milestones for completion along the patient pathway. The patient pathway progress indicator 300 extends from a first step of being enrolled in the remote patient monitoring program at step 302 through various steps of the process including patient onboarding at step 304, initial baseline surveys completed 306, RDN recommendation viewed by patient 308, RDN interventional cardiologist (IC) consult scheduled 310. RDN recommendation sent to IC 312, RDN consultation 314, insurance authorization 316, RDN procedure scheduled 318 (not shown) and RDN procedure performed 320 (not shown). The patient's progress along the patient pathway 300 may include a check mark identifying items that are complete and a date of completion. As will be appreciated, more or fewer milestones may be depicted on the patient pathway indicator 300 depending on the patient's individual pathway. A most recently completed step may be highlighted to indicate where along the pathway the patient is currently situated. Links 322 can allow for entry of data, such as date of completion, or transmission of a request (e.g., that a patient reviewed materials or to schedule a consultation). The information regarding the completion of each milestone of the patient pathway progress may be input manually by the clinician, their staff, the patient, or automatically updated to reflect data in the patient's EMR or other activities on the platform that indicate a milestone has been completed.

Following the patient pathway, patients identified as candidates for RDN therapy undertake this pathway in concert with the medical professionals at the HTN Clinic and the IC ensuring communication discussions at each step between the medical professionals and the patient. In this way the medical professionals can provide information, as well as prompts for the next steps to be undertaken. Further, by collecting and aggregating the patient blood pressure, medication adherence, and quality of life survey data, both the medical professionals at the HTN Clinic and the IC can be provided useful, sorted, and relevant data in a form that is most actionable to the IC ins assessing the ultimate candidacy of the patient for RDN therapy.

The actions of the HTN Clinic in assisting the patient in achieving referral to an IC for RDN therapy are performed simultaneously with the remote monitoring of those very same patients. This allows the HTN Clinic to ensure that the patients are in compliance with both their blood pressure monitoring and medication adherence regardless of whether they are ultimately deemed a candidate and undergo denervation or not. By combining the highly granular data collected via remote monitoring, such as never could be achieved by in office data collection, a much greater insight into the patient, their disease state, their medication and diet compliance capacity, and additional health data (e.g., activity tracking) a more robust data analysis can be undertaken to ensure that those that are candidates for RDN therapy are provided an opportunity to consult with an IC, while allowing the HTN Clinic to focus their attention on the patients who are not candidates for RDN therapy or who require a more hands on approach in order to achieve medication and diet compliance (e.g., traditional in-office data collection) and combinations of remote and in-office data collection.

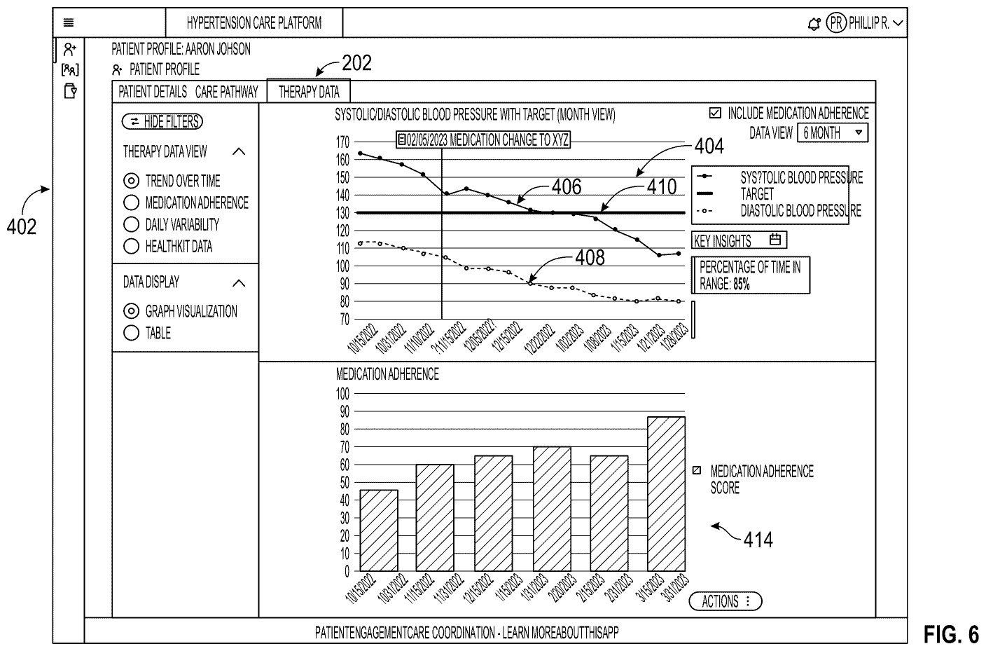

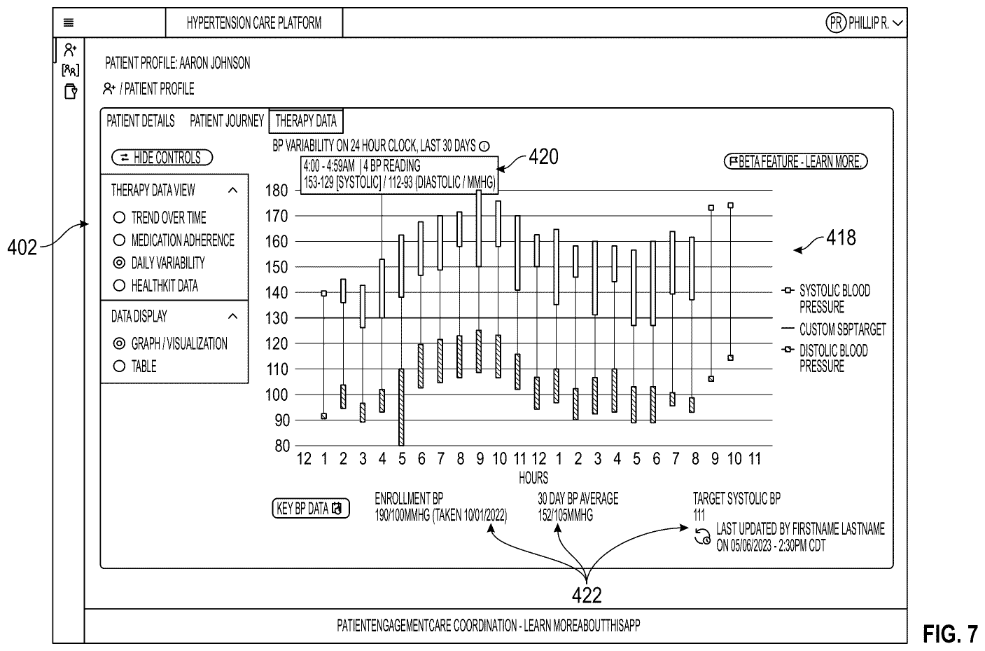

Referring to FIG. 5 a “Therapy Data” tab 202 can be selected. A variety of cultivated patient data may be presented to allow a medical professional to analyze the patient's vital signs and other reported data to the HTN Clinic. FIG. 6 depicts one representative user interface 402, graph 404 displays the systolic blood pressure trend data 406 and diastolic blood pressure trend data 408. In addition, a target line 410 may be displayed to provide a comparison point for the trend data. The graph 404 may be selected from a list 412 including for example a trend over time (as shown), medical adherence as displayed in field 414, daily variability (FIG. 7), HealthKit data (not shown), and others. Table 104 allows for the data to be displayed as a bar graph (e.g., in field 414) or as a plot (e.g., graph 404). Those of skill in the art will recognize that the health kit data is just the data as received from the patient's remote monitoring application (described below) in its raw or substantially unprocessed form. The HealthKit and other aspects of the data from the remote monitoring of the patient are described in greater detail below.

FIG. 7 displays a graph 418 displaying the daily variability of blood pressure data. In contrast with traditional in-office visits, the use of remote monitoring and self-reporting allows for the receipt of multiple daily readings of the patient's vital signs as well as readings at differing times of the day. As described above, a patient's blood pressure may vary greatly throughout the day. This variability is another reason individual once per week or even twice per week in-office measurements are insufficient to truly understand a patient's blood pressure related health. The graph 418 in FIG. 7 displays the range of blood pressure readings received over the course of the last 30 days, at various times of the day. As shown, the patient in question has taken blood pressure readings at most times of the day, though not many between 9 pm and 2 am, with none recorded between 11 and 12 p.m. At other points such as 4:00 am a total of four separate blood pressure readings have been acquired over the past 30 days. As will be appreciated there are a variety of reasons for variability in collection of data by the patient ranging from variability in daily schedule to a patient wanting to assess their own blood pressure based on how they feel at a particular moment during the day. By plotting this data over the course of 30 days, the variability can be observed, both from time to time during the day, and the variability at any particular time. By selection of one of the times (e.g., 4:00 a.m.) a pop-up 420 displays the number of readings taken at that time over the last 30 days as well as the variability of the blood pressure readings (both systolic and diastolic) within that time window (4:00 a.m.-4:59 a.m.). Other data may be presented in fields 422 including a blood pressure acquired at enrollment, a 30-day blood pressure average, and target blood pressure values, as well as others that can be useful or relevant in assessing the health of the patient.

As described above, a further aspect of the disclosure is directed to an application, downloadable to a patient's smartphone, that is configured to capture vital sign information and periodically connect to the application, described above in connection with FIGS. 1-7, above at the HTN Clinic. Additionally or alternatively, the smartphone application may connect to a cloud-based system for collecting and analyzing the data or to other third-party system so that the blood pressure data, medication compliance data, vital sign data, and patient data can be accessed and assessed in other medical computing systems.

Most vital sign data collected by an application on a smartphone can share that vital sign information with a second application. Thus, in one aspect of the disclosure, the application receives the vital sign data directly from a device (e.g., a blood pressure cuff or weight scale) that may be in communication with the application for example via BLUETOOTH or other wired or wireless communication protocol. The application may also pull or be pushed vital sign data from other applications such as the APPLE® HealthKit. Further, the activity data of the patient can be collected either from fitness devices or applications on the patient's smartphone. Still further, various cloud services may be employed to manage the transfer of data from a device to the application(s) described herein. This could include scenarios where the device communicates with a third-party cloud service and the application(s) receives the vital sign data from the third-party cloud system. Additionally or alternatively, the third-party could system may allow for the data received from the device to be copied to a cloud based system associated with the application(s) and the vital sign data is accessed by the application(s) from their associated cloud system. These enable tracking of lifestyle modifications of the patients, providing further insight into how the patient actually lives.

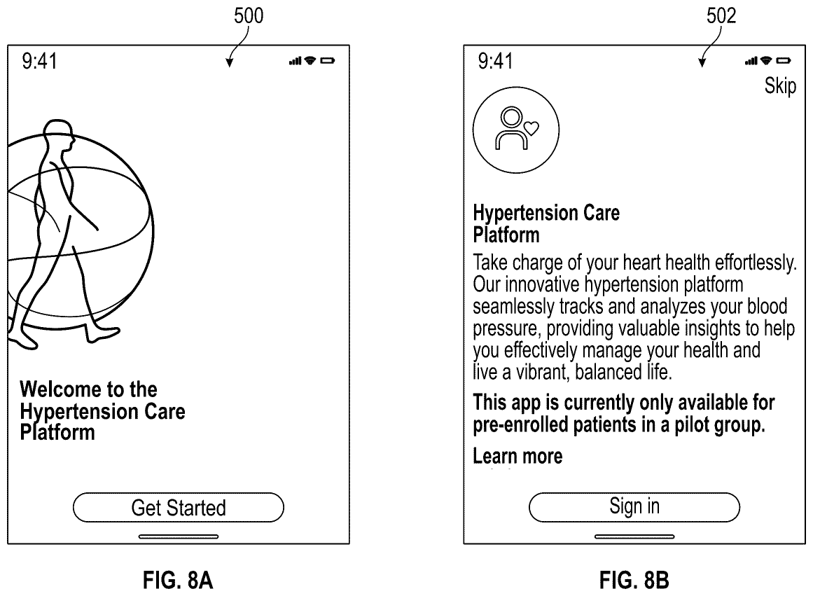

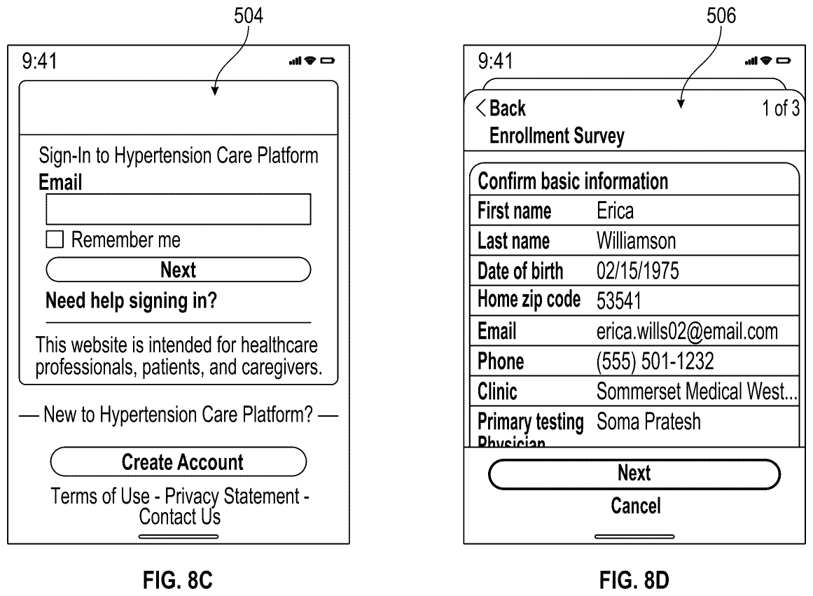

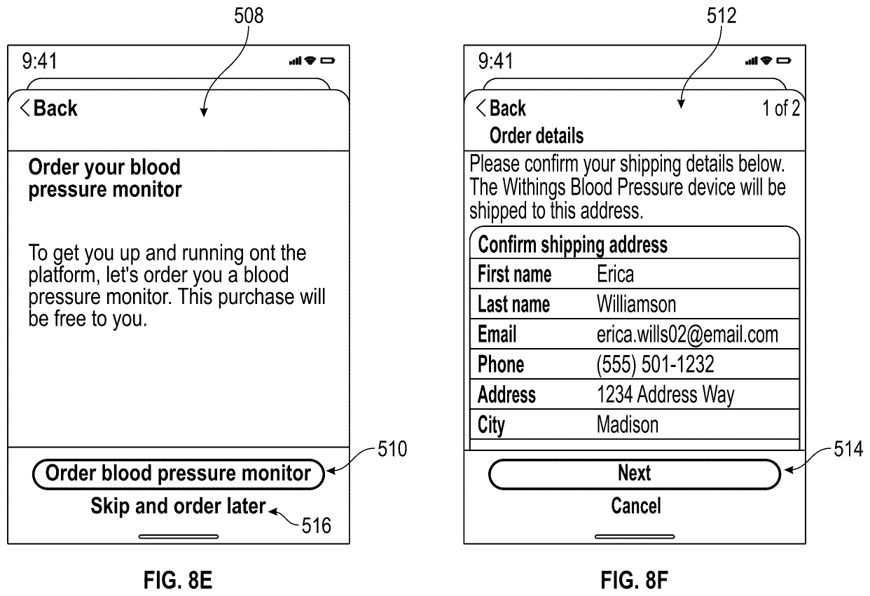

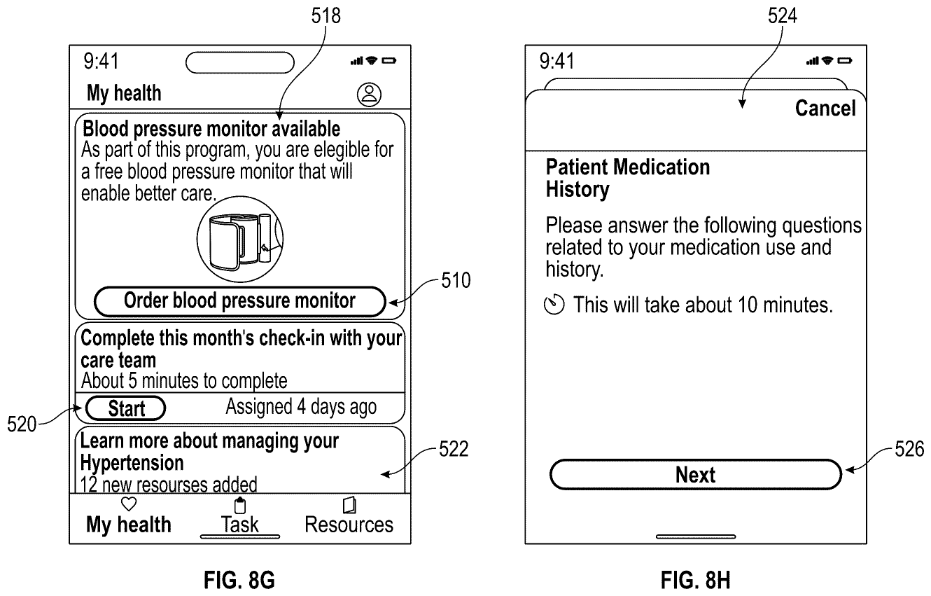

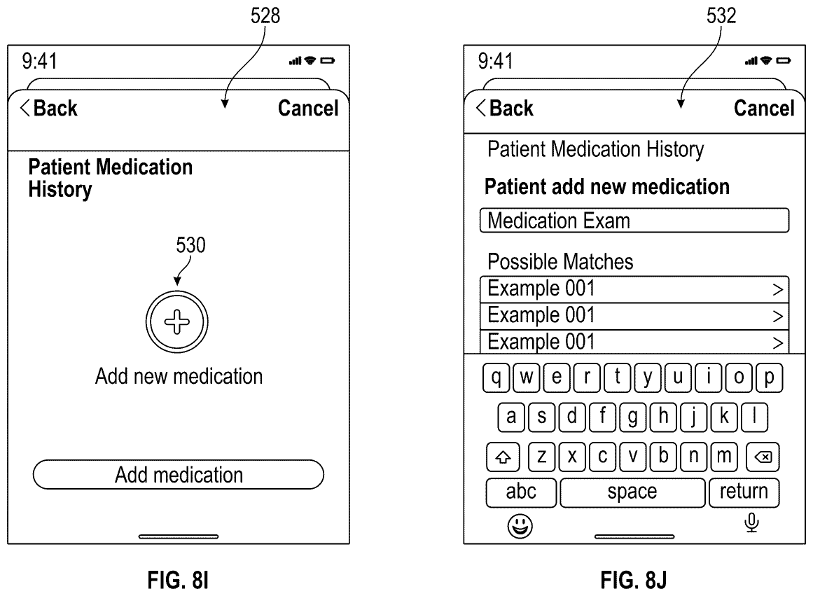

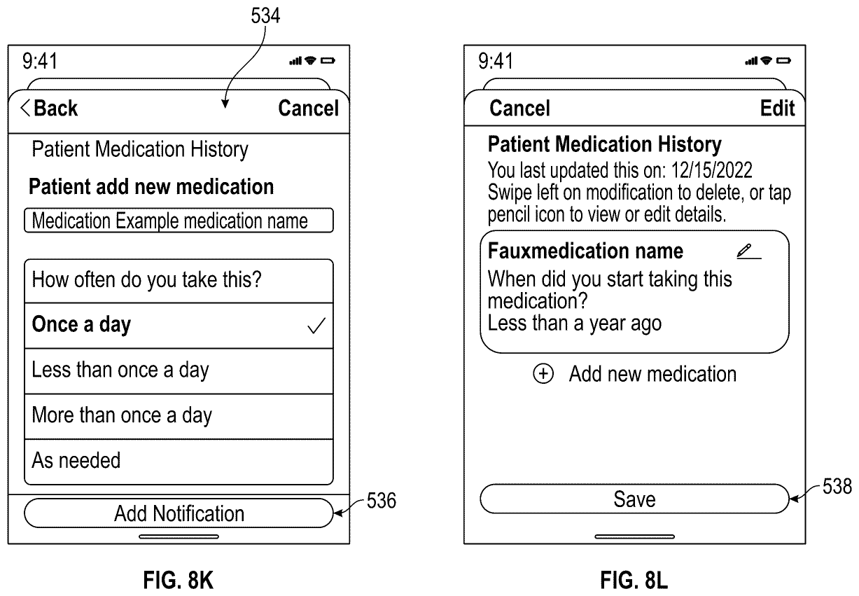

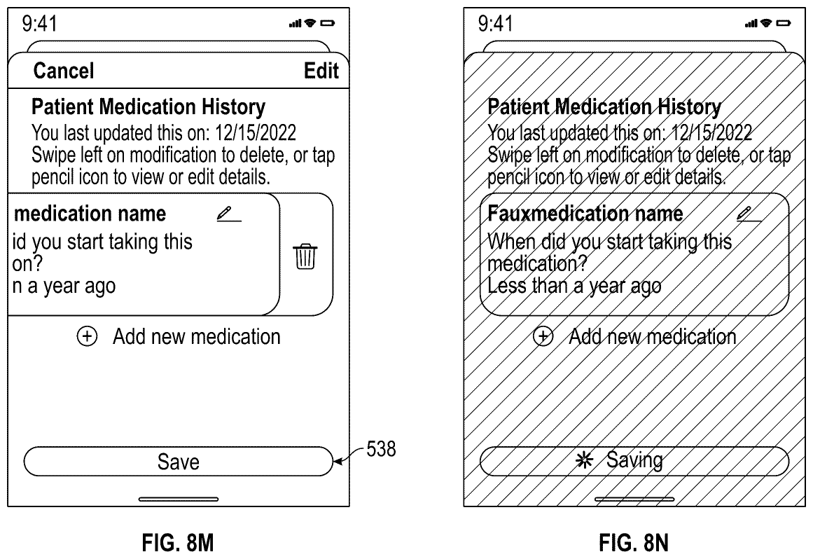

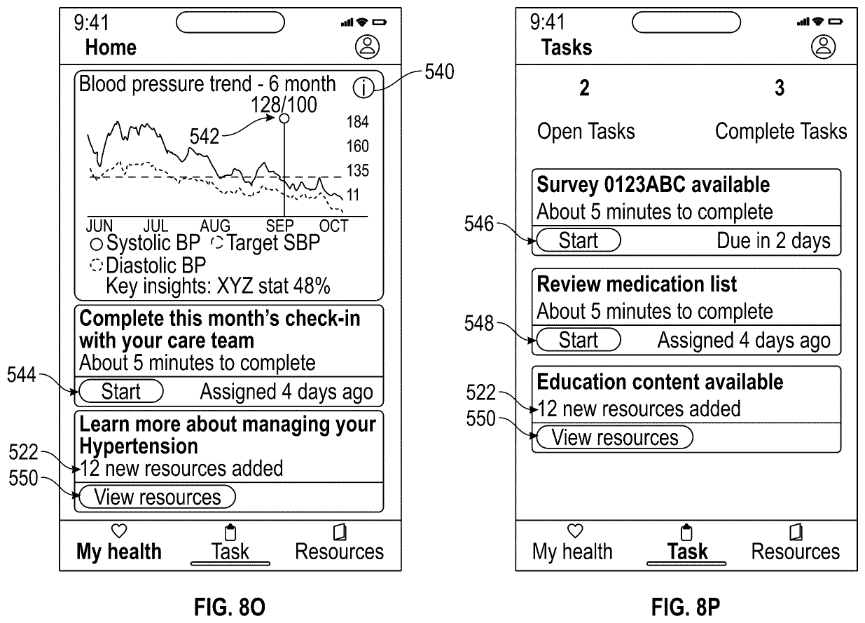

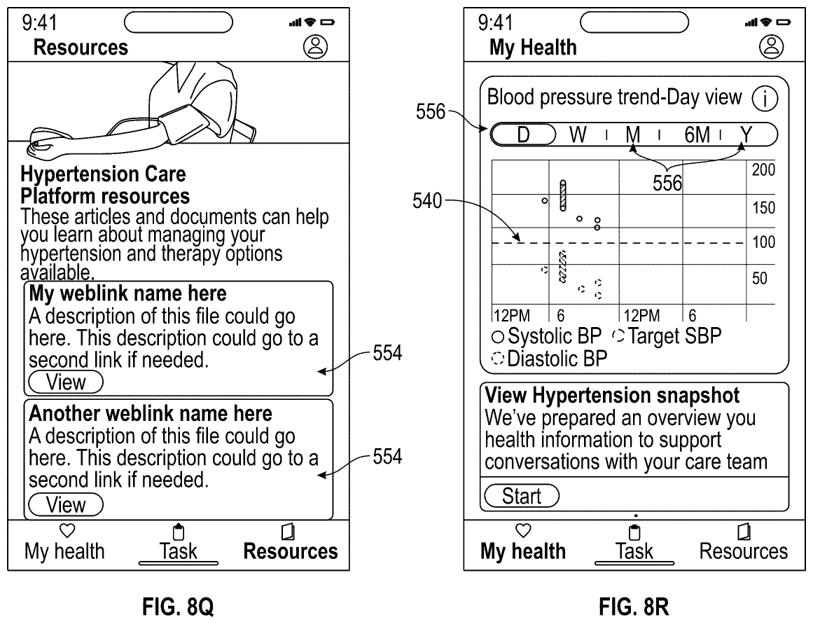

FIGS. 8A-Q depict screens in a remote patient monitoring application as they appear on the patient's smartphone. FIG. 8A depicts an initial screen 500 following download of the application from an app store or another source. FIG. 8B presents a sign-in invitation screen 502 as might appear each time the patient accesses the application, or in some instances just the first time the patient signs in. FIG. 8C depicts a sign-in screen 504, where the sign-in information is collected by the application, or the patient may create an account if they have never signed in to the application.

As part of an initial enrollment, the patient is requested by the application to undertake an enrollment survey as depicted in screen 506 of FIG. 8D. The patient may be asked to complete the survey, or if they have already established an account, for example via the application at the HTN Clinic, and signing in to the application on the smartphone for the first time, the patient may be asked to merely confirm the data which is pre-populated. At screen 508, in FIG. 8E, if not already completed, the application may assist the patient in ordering an appropriate blood pressure monitor device. By selecting tab 510, screen 512 in FIG. 8F is displayed and order details are confirmed. By selection of the next button 514, the order is placed. If, however, in screen 508 the skip link 516 is selected, screen 518 in FIG. 8G is displayed. As can be seen, the patient can at any time order the blood pressure monitor by selecting tab 510.

Also shown in FIG. 8G is survey tab 520 and an informational tab 522. Selection of survey tab 520 launches a self-reported survey such as a medication adherence survey or a quality-of-life survey. These surveys provide self-reported data that is useful to the medical professionals at the HTN Clinic, in analyzing the vital sign data (e.g., blood pressure data) being remotely monitored and transmitted to the HTN Clinic for review. One example of a survey for the patient to complete include medication adherence surveys where the patient self-reports whether they have adhered to their medication's prescribed routine (e.g., every day, multiple times per day, etc.) and the occurrence of any “medication holidays” where the patient either could not or did not take the medication in the prescribed amounts or at the prescribed frequencies. A second survey is a quality-of-life survey in which the patient self-reports on their current situation, whether there have been any falls or other incidents the medical professional at the HTN Clinic needs to be aware of, changes in diet, mobility, etc.

Selection of the informational tab 522 provides access to the patient of a variety of informational material for their review. This may include material related to managing their blood pressure as well as information regarding RDN therapy that they are required to review prior to progressing on the patient pathway (described above). Confirmation of the review of these materials may be reported back to the HTN Clinic via the communications functionality of the application on the smartphone (e.g., via the internet).

FIGS. 8H-8N enable addition of medication data. FIG. 8H is a landing screen 524 to start the process of adding medication history. By selection of the next button 526 screen 528 of FIG. 8I is displayed. Selection of the add new medication link 530 launches a fillable form 532 as shown in FIG. 8J where the patient can enter the medication name. Once added, FIG. 8K is displayed where the frequency of taking the medication can be selected from table 534. Selection of the add medication button 536, adds the medication to the patient medication history, as shown in FIG. 8L. FIG. 8M allows for a selected medication to be deleted (e.g., in case of error, or after cessation of taking the medication). Once the patient's medication history is complete, the save button 538 may be selected and the medication history data is saved to the application on the smartphone (See FIG. 8N) and transmitted to the application at the HTN Clinic. As will be appreciated, this data should already be available at the HTN Clinic, and one or more algorithms on the application at the HTN Clinic can review the entered medication history for errors or omissions and alert the medical professionals at the HTN Clinic, or the patient directly via the application on the smartphone. In addition, as many patients have multiple comorbidities and receive prescriptions from doctors outside of the HTN Clinic, entry of the medication history allows for review and analysis to determine whether there are any potential negative interactions of the medications that could be affecting the patient or their blood pressure readings.

Once enrolled with medication history entry completed and the blood pressure monitor ordered the patient can begin to provide their remotely monitored blood pressure to the HTN Clinic. FIG. 8O is a home screen of the remote patient monitoring application. In FIG. 8O, a graph 540 of the patient's blood pressure is presented. The X-axis of graph 540 may be selected to present information relevant to the patient (e.g., one week, two weeks, one month, two months, three months, six months, one year, or intervals between any of these). The Y-axis of the graph are the actual blood pressure readings of the patient. Two trend lines are depicted separately, the systolic blood pressure and diastolic blood pressure. A target blood pressure may also be depicted. A marker 542 can be moved across the graph, and the intersection of the marker 542 and the trend lines is displayed and indicates the actual reading that is part of the two trend lines. In FIG. 8P the marker intersects the trend lines at 128 (systolic blood pressure) over 100 (diastolic blood pressure). Though graph 540 depicts the blood pressure trend line data, the disclosure is not so limited and graph 540 may also or additionally display the medical adherence graph (FIG. 6) or the daily variability of blood pressure data (FIG. 7) as well as other relevant data regarding the patient's blood pressure health without departing from the scope of the disclosure.

In FIG. 8O, the patient may initiate a “check-in” with the HTN Clinic by selecting tab 544. Selection of tab 544 launches the screen seen in FIG. 8P, where tasks for completion by the patient are presented. For example, the HTN Clinic may request a medication adherence survey and quality of life survey be completed by the patient at regular intervals (e.g., weekly or monthly). These surveys may be presented in tab 546. In addition, the HTN Clinic may require a regular review of the patient's medication list. These may include assessment of medication adherence (e.g., are they regularly taking their medication), self-reporting of accidents or injuries, self-reporting of excessive alcohol consumption during the reporting period, utilization of healthcare services, lifestyle modifications, and other questions. Selecting tab 548 brings the user back to FIGS. 8H-N where the patient can utilize the functionality described above to add or delete medications as appropriate.

Referring back to FIG. 8O, tab 550 provides access to resources for the patient's review. These educational resources include, for example, websites and articles that assist the patient in understanding their condition, provide tips on improving their condition, and other aspects of managing their life. Tab 550 is also available for the patient to select from the screen on FIG. 8P, in both FIGS. 80 and 8P tab 550 may include an indicator 552 of new or as yet un-reviewed resources. When tab 550 is selected either in the screen of FIG. 8O or FIG. 8P, the screen of FIG. 8Q is presented on the patient's smartphone. These resources may each have their own tab 554 for the patient to select and review.

FIG. 8R depicts an alternative graph 540 of the patient's blood pressure data. Unlike FIG. 8O where a 6-month graph of the patient's blood pressure data is depicted FIG. 8R depicts a single day's daily blood pressure data (both systolic and diastolic blood pressure) and a target systolic blood pressure. Alternative lengths of time may be selected by the patient using one of the tabs 556 (e.g., day, week, month, six-month, year).

As will be appreciated, the remote patient monitoring application may include notification functionality such that requests of the patient may be made by the HTN Clinic through their application, and the patient receives an alert indicating a request has been received. Further, in accordance with the disclosure, the application at the HTN Clinic may be configured to analyze received blood pressure and other vital sign data and issue one or more alerts to the remote blood pressure monitoring application including “contact the HTN Clinic, or “go to the Emergency Room,” etc.

In addition, the notifications may be in conjunction with questions to be answered periodically by the patient directly. As noted above, all this data, which largely comports with the data that the HTN Clinic would capture during a scheduled appointment, can be pushed to the application at the HTN Clinic. Alternatively, the data can be pushed to a cloud-based system for collecting and analyzing the data or to another a third-party system. Regardless of the path, the data can be incorporated into the EMR, or other data files used by the HTN Clinic to store patient data and make clinical assessments of the patients.

As noted above, a cloud-based system may be employed to collect patient data (e.g., vital sign data), store the patient data, and undertake the analyses described herein. The remote patient monitoring application may be configured as a gateway pulling data from other applications or receiving it from connected devices (e.g., blood pressure monitor, scale, etc.) and pushing of that data to the cloud-based system. In one aspect of the disclosure, the algorithms (e.g., neural networks, AI, and others) described in connection with the application at the HTN Clinic are cloud-based. The cloud-based applications may communicate directly with the patient via the remote patient monitoring application based on the analysis of the data. The cloud-based applications may also be accessible via the application at the HTN Clinic enabling the alerting of the HTN Clinic or another medical professional based on the cloud-based application's analysis of the data (e.g., dramatic change in blood pressure or vital signs, a life-threatening condition, etc.).

Though described in aspects herein in connection with a determination of eligibility for an RDN procedure, the disclosure is not so limited, and the HTN Clinic application and the remote patient monitoring application can be utilized by the HTN Clinic and the patient even if the patient does not qualify for, or is not interested in pursuing, a denervation procedure. As will be appreciated the collected data can be used in the optimization and adjustment of a patient's treatment plan and in triaging patients for treatment modification and other purposes without departing from the scope of the disclosure.

Once a patient is enrolled in the remote patient monitoring application (as described in connection with FIGS. 8A-8Q), patient data including blood pressure, vital sign, medication adherence, and survey is collected and shared with the application at the HTN Clinic and presented to medical professionals (See e.g., FIGS. 1-7). FIGS. 5A-5C, described above, include a patient pathway field 216. Depending on the data collected from the patient, potential pathways are presented to the medical professionals at the HTN Clinic in the patient pathway field 216. As described above a variety of patient pathways 216 can be presented. For example, to be in consideration for an RDN procedure the patient must satisfy some initial criteria (e.g., blood pressure >140/90 mmHg, primary hypertension, three or more blood pressure reduction medications, inability or unwillingness to adhere to medication regimen, elevated cardiovascular risks, or others). If the patient is found to be initially eligible, then the patient can start on their patient pathway, and have their progress reflected in the patient pathway progress indicator 300.

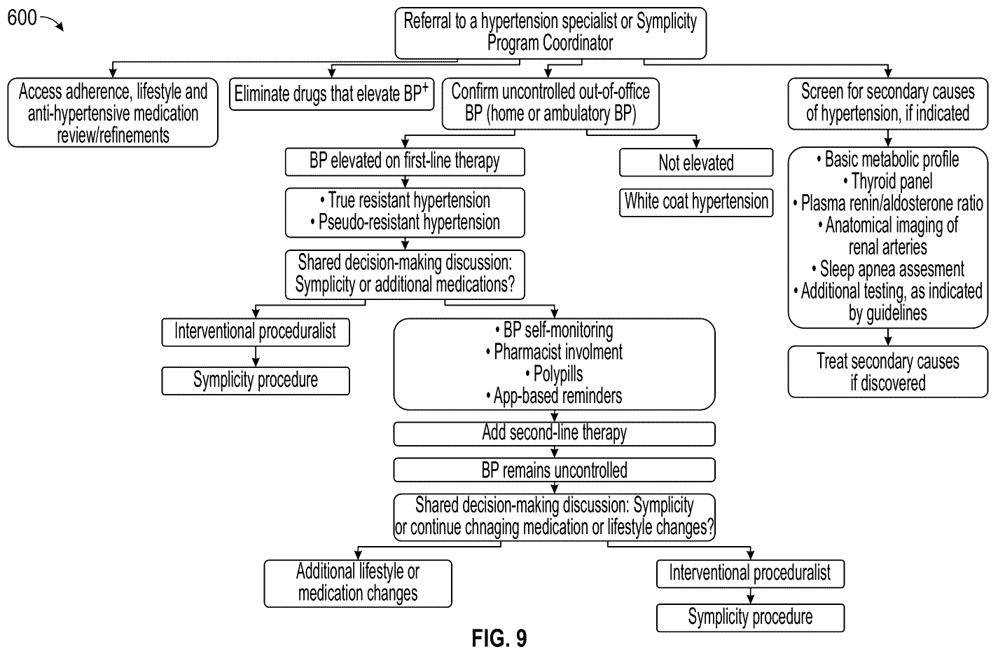

FIG. 9 depicts a decision tree 600 as might be employed by an HTN Clinic in concert with the IC and the patient to confirm that the patient expressing one or more of the initial criteria is in fact a candidate for an RDN procedure. In accordance with aspects of the disclosure, many of the steps in the decision tree can utilize the self-reported data from the remote patient monitoring application and utilizing algorithms in the HTN Clinic application (e.g., neural networks or AI) provide refined or actionable data for the HTN Clinic medical professionals and the IC to assess, confirm, and counsel the patient.

Not all hypertension patients are good candidates for the RDN procedure. In some instances, their condition may be better alleviated by changes in behavior (e.g., smoking cessation, alcohol use reduction, diet changes, exercise, reduction of work-related stresses, elimination of blood pressure elevating drugs, etc.). Some patients have comorbidities that make them ineligible for an RDN procedure. These factors can be assessed manually by the medical professionals at the HTN Clinic, or in some instances an algorithm can analyze a variety of data sources (e.g., the EMR, survey data. Etc.) and provide indicators or summaries for use by medical professionals. As will be appreciated, for patients not currently eligible for denervation, that status may change as management of their disease progresses, additional clinical data is generated, and regulatory approval received for treating such patients, or the like.

One aspect of the decision tree is the confirmation of the uncontrolled out-of-office high blood pressure. The remote patient monitoring application is uniquely suited for providing this data to the HTN Clinic without unnecessarily inconveniencing the patient or the HTN Clinic staff utilizing the systems and methods described above. Once it is confirmed that the patient does not suffer from “white-coat hypertension” and does not have a secondary cause of their high blood pressure, the HTN Clinic medical professionals can provide the patient with information regarding RDN procedures and undertake a consultation with the patient. Where a patient is motivated, and the HTN Clinic medical professionals agree on the benefits of an RDN procedure, the patient can be referred to an IC. The IC can be provided with the relevant patient data from the HTN Clinic application or given access to the patient's data via an IC practice version of the HTN Clinic application. Following consultation with the IC, and acceptance by the IC that the RDN procedure is indeed appropriate for the patient, the patient can continue to follow the patient pathway progress indicator 300, schedule their RDN procedure, and receive the RDN therapy.

Alternatively, where a patient is not quite ready for the RDN procedure or the HTN Clinic medical professionals counsel towards other options, no referral is needed, and remote monitoring of the patient, as described above, can continue. Optionally additional therapies can be employed. Where the patient's blood pressure remains largely uncontrolled despite the initial and additional therapies, a further consultation between the patient and the HTN Clinic medical professional can be undertaken to determine if the RDN procedure is now appropriate. If it is decided by the patient, in consultation with the HTN Clinic medical professional, that the patient is now ready for the RDN procedure, the patient is referred to an IC. Following consultation with the IC, and acceptance by the IC that the RDN procedure is indeed appropriate for the patient, the patient can continue to follow the patient pathway progress indicator 300 and can schedule their RDN procedure. Alternatively, additional medications or lifestyle changes may be prescribed where the patient and/or the HTN Clinic does not believe the RDN procedure is in the patient's best interest at that time. The remote patient monitoring application can continue to provide patient blood pressure and vital sign data as described herein.

Following the RDN procedure, the IC may, via the remote patient monitoring application, be alerted to any postprocedural complications observed in the patient data (e.g., spikes in blood pressure, etc.). The IC may also generate a post-procedure survey for completion by the patient to inquire regarding other aspects of the procedure (e.g., wound healing, pain, etc.). If there is an issue in the post-procedural data, the software system of the IC can generate an alert so that appropriate mitigation efforts can be undertaken. If there is no issue in the post-procedural data, no actions need to be undertaken. Upon a successful denervation procedure, the expectation is that the hypertension (blood pressure) will be reduced, and the patient's other symptoms will ameliorate over time.

Still further, periodically after the procedure a post-operative snapshot detailing the changes in patient blood pressure (and other relevant data) as compared to pre-procedural data can be generated (e.g., by the HTN Clinic application or an IC practice version of the HTN Clinic application). This post-operative snapshot can be utilized by the IC in assessing the performance of the procedure overtime and assist the IC with future patient consultations.

In some instances, the patient continues to use the remote patient monitoring application and having their blood pressure and other vital signs acquired and pushed HTN Clinic application for monitoring. At some point the patient's condition may improve such that the medical professionals at the HTN Clinic can be determined to be completely treated and the patient may stop use of the remote patient monitoring application. Alternatively, based on the results of the denervation procedure the frequency of patient interactions with the HTN Clinic can be reduced as well as the frequency of self-reporting of patient vital sign data as their condition improves.

As noted above, in accordance with the disclosure, some or all of the steps of the decision tree depicted in FIG. 9 may be performed, at least initially, by one or more algorithms (e.g., neural networks or AI) operating on or in conjunction with the application at the HTN Clinic to identify patients that are candidates for an RDN procedure. The algorithms can analyze all of the patient data including EMR data, self-reporting survey data, blood pressure data, medication adherence data and other data to summarize the data into a more useable form for the HTN Clinic medical professionals to make determinations along the decision tree of FIG. 9.

As noted above, following initial enrollment, in connection with the patient pathway progress indicator 300, the patient may be provided with a variety of educational materials via (e.g., via the tabs 554 (FIG. 8Q) in the remote patient monitoring application). These materials include the standard description of care, the potential for side effects, and other materials regarding denervation and, optionally, other aspects of treatment of hypertension.

In accordance with a further aspect of the disclosure, once the patient is referred to the IC, the remote patient monitoring application may be connected to push the recorded data to the IC (e.g., via the HTN Clinic application or an IC practice version of the HTN Clinic application). The IC is thus provided the data where the readiness of the patient or the appropriateness of denervation for the patient is continually assessed as part of a shared decision-making process with the patient. As will be appreciated, the patient data (e.g., blood pressure or vital signs) enables the IC to undertake patient triage so that patients, having been accepted by the IC for an RDN procedure, can be appropriately scheduled for the RDN procedure. In some instances, the triage is based on the urgency of need of the patients, however, other factors may also be considered by the IC in making triage determinations. Following the triage determinations, the patient may be scheduled for the denervation procedure, and the denervation procedure is subsequently undertaken. With the denervation procedure undertaken, and following the typical recovery period, the patient is released from the hospital or surgical clinic where the procedure was undertaken.

As noted above, the patient data (e.g., blood pressure and vital sign data) can also be analyzed and assessed by one or more cloud-based systems in communication with the remote patient monitoring application or the HTN Clinic application. The cloud-based system may conduct the analysis and forward the analysis to the HTN Clinic application or one or more software system of the IC. Still further, the IC staff may analyze the patient data, either solely or to review and approve the analysis of the software system or the cloud-based system. The triage may be assessed on a daily basis or on some other periodic basis (e.g., the frequency of reporting such as weekly). Software applications can analyze the reporting data and detect changes in conditions or trends of which the IC staff should be alerted. In such instances, the staff of the IC can reach out to the patient to determine what might have caused the change in vital signs. As will be appreciated, the patient's data remains available to the HTN Clinic with the application at the HTN Clinic so that the patient can continue to be monitored by the HTN Clinic all through the involvement of the IC with the patient. The data from all the referred and/or post-procedure patients may be available to the IC and can be analyzed and assessed for practice level reporting. This includes determination of patterns in both successful and unsuccessful patients to inform the patient selection process and further refine the patient and clinician surveys as well as the triage modeling of the patients, and potentially the parameters of the therapy applied to the patient.

Those having skill in the art will recognize that the RDN procedure may employ one or more of monopolar RF, bi-polar RF, microwave, ultrasound, focused ultrasound, cryogenic, chemical, pulsed field ablation (PFA), and/or other modalities. Similarly, while often considered in the context of application of therapy to the renal artery to denervate sympathetic or, in certain embodiments, parasympathetic, nerves in, around, and near the renal arteries, the present disclosure is not so limited and can be employed for denervating nerves accessible via any blood vessel (e.g., celiac trunk, hepatic, splenic, gastric, superior mesenteric, inferior mesenteric, gonadal, splanchnic, etc., and branches and/or combinations of each) of other luminal tissue (e.g., a bile duct).

Still further those of skill in the art will recognize that any assessment steps (e.g., of FIG. 9) described as performed herein as being performed by an application of software system may be merely suggestions for proceeding offered by the application or software system and the ultimate determinations may be made by a clinician such as a doctor or nurse after reviewing the suggestions from the application or software system.

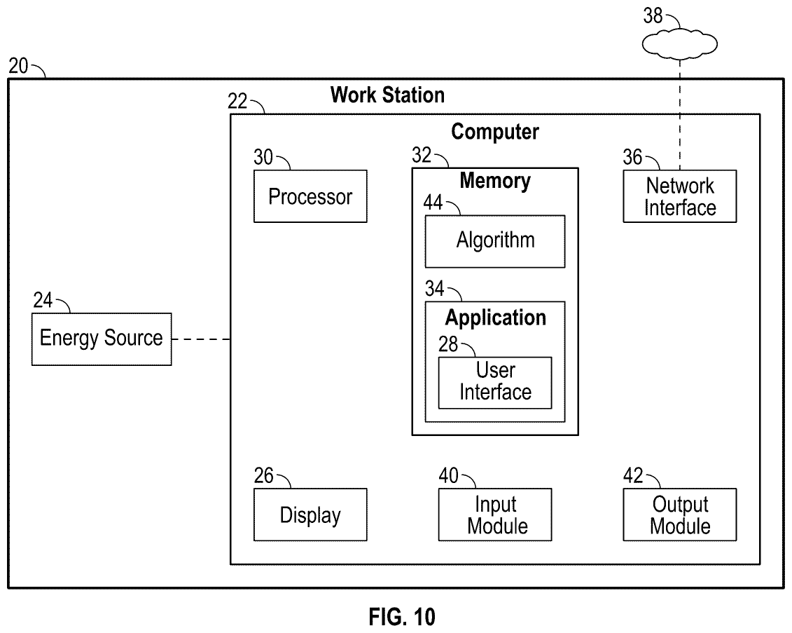

In accordance with FIG. 10, the software systems described herein may be embodied in one or more computing devices and configured to receive patient data (e.g., blood pressure or vital sign data). In some instances, the computing device is configured to receive patient data from an application on the patient's smartphone or from a cloud-based system (described above). Similar components enabling the collection of patient data (e.g., blood pressure) from a connect device (e.g., a blood pressure monitor) and transmission to one or more computing devices at the HTN Clinic, the IC. The computing device 22 may be a desktop computer, a laptop computer, a tablet, a smartphone, or another computing device with display 26. The computing device 22 includes a processor 30 which executes software stored in a memory 32. The memory 32 may store one or more applications 34 and/or algorithms 44 (e.g., neural network or AI) to be executed by the processor 30. A network interface 36 enables the workstation 20 to communicate with a variety of other devices and systems (e.g., via Bluetooth® or via the internet). The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, and/or videos may be stored. The cloud storage system 38 may be remote from or on the premises of the hospital such as in a control or hospital information technology room. It is envisioned that the cloud storage system 38 could also serve as a host for a variety of patient data including EMR data, acquired images (e.g., fluoroscopic, computed tomography (CT), magnetic resonance imaging (MRI), cone-beam computed tomography (CBCT), etc.), and other data. An input module 40 receives inputs from an input device such as a keyboard, a mouse, voice commands, an energy source controller (e.g., a foot pedal or handheld remote-control device. An output module 42 connects the processor 30 and the memory 32 to a variety of output devices such as the display 26. In embodiments, the display 26 may be a touchscreen display.

Although described generally hereinabove, it is envisioned that the memory 32 may include any non-transitory computer-readable storage media for storing data and/or software including instructions that are executable by the processor 30 and which control the operation of the workstation 20. In an embodiment, memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 20.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

I claim:

1. A system for remote monitoring a patient comprising:
a smartphone including a memory and a processor, the memory storing therein an application that when executed by the processor performs steps of:
receiving patient blood pressure data from a blood pressure monitor;
transmitting the patient blood pressure data to a medical professional's application;
receiving one or more tasks from the medical professional's application; and
transmitting results of the one or more tasks to the medical professional's application.

2. The system of claim 1, wherein the application when executed by the processor receives vital sign data of the patient from a third-party application.

3. The system of claim 2, wherein the third-party application is a healthkit application configured to receive vital sign information from one or more connected devices.

4. The system of claim 1, wherein the application when executed by the processor receives and presents educational materials for the patient in a user interface on the smartphone.

5. The system of claim 4, wherein the application when executed by the processor transmits an indication of review of the educational materials to the medical professional's application.

6. The system of claim 1, wherein the application when executed by the processor presents the received blood pressure data in a user interface on the smartphone.

7. The system of claim 1, further comprising a computing device including a memory and a processor, the memory storing therein the medical professional's application that when executed by the processor monitors the patient based on received results of the one or more tasks and received patient blood pressure data.

8. The system of claim 7, wherein the medical professional's application when executed by the processor determines at least in part by using or more of an algorithm, a neural network, or an artificial intelligence (AI), a patient's suitability for a denervation procedure.

9. The system of claim 8, wherein the medical professional's application when executed by the processor employs one or more of an algorithm, neural network, or AI analyze the received results of the one or more tasks, the received blood pressure data, and an electronic medical record of the patient to determine suitability for a denervation procedure.

10. The system of claim 8, wherein the medical professional's application when executed by the processor generates a referral of the patient to an interventional cardiologist (IC) for assessment of the patient for the denervation procedure.

11. A method for remote monitoring of a hypertension patient comprising:
enrolling a hypertension patient in a remote patient monitoring application configured for a smartphone of the patient;
receiving by the remote patient monitoring application patient blood pressure data from a blood pressure monitor;
transmitting via the remote patient monitoring application the patient blood pressure data to a medical professional's application;
receiving via the remote patient monitoring application one or more tasks from the medical professional's application; and
transmitting results of the one or more tasks via the remote patient monitoring application to the medical professional's application.

12. The method of claim 11, further comprising receiving via the remote patient monitoring application educational materials; and
transmitting to the medical professional's application and indicator that the educational materials have been reviewed.

13. The method of claim 11, further comprising presenting a graph of the patient's blood pressure data on a screen in the remote patient monitoring application.

14. The method of claim 11, further comprising monitoring at the medical professional's application the patient based on the received results of the one or more tasks and the received patient blood pressure data.

15. The method of claim 14, further comprising determining via the medical professional's application suitability of the patient for a denervation procedure.

16. The method of claim 15, wherein the determining of suitability of the patient for a denervation procedure is made by hypertension clinic (HTN Clinic) personnel.

17. The method of claim 15, further comprising generating via the medical professional's application a referral of the patient to an interventional cardiologist (IC) for assessment of the patient for the denervation procedure.

18. The method of claim 15, wherein the determining of suitability of the patient is for the denervation procedure is performed at least in part by one or more of an algorithm, a neural network, or an artificial intelligence (AI).

19. The method of claim 18, wherein the one or more of an algorithm, neural network, AI analyze the received results of the one or more tasks, the received blood pressure data, and an electronic medical record of the patient.

20. The method of claim 15, further comprising transmitting via the remote patient monitoring application blood pressure data to the medical professional's application after performance of a denervation procedure.
